(19) United States
(12) Patent Application Publication
(10) Pub. No.: US 2026/0308574 A1
(43) Pub. Date: Oct. 8, 2026

(54) METHOD

(71) Applicant: SANIA TX LTD, London (GB)

(72) Inventors: Andrew MURRAY, London (GB); Alba GUIJARRO BELMAR, London (GB)

(73) Assignee: SANIA TX LTD, London (GB)

(21) Appl. No.: 19/481,519

(22) PCT Filed: May 13, 2024
(86) PCT No.: PCT/IB2024/054634

§ 371 Date: Nov. 4, 2025

(30) Foreign Application Priority Data
May 16, 2023 (GB) 2307263.0

(51) Int. Cl. C12N 15/10; C12N 15/86; C12Q 1/686

(52) U.S. Cl. CPC C12N 15/1086; C12N 15/1082; C12N 15/1093; C12N 15/86; C12Q 1/686; C12N 2750/14143; C12N 2840/203

(57) ABSTRACT

The present disclosure relates to a novel screening method to screen multiple pGREs in parallel and to nucleotide construct for use in the method The constructs comprising nucleotide blocks, in sequence PRIMREG1-pGRE-PRIMREG2-GENE, or GENE-PRIMREG1-pGRE-PRIMREG2 wherein PRIMREG1 is either a primer recognition sequence 1, or a FLEx cassette comprising a primer recognition sequence 1. pGRE is a putative gene regulatory element, PRIMREG2 is either a primer recognition sequence 2, or a FLEx cassette comprising a primer recognition sequence 2, GENE comprises nucleotide blocks REPSEQ, IRES and SSR, wherein REPSEQ is a reporter gene and is either present or absent, IRES is either an internal ribosome entry site or a 2A self-cleaving peptide, and SSR is a site specific recombinase gene. At least one of PRIMREG1 and PRIMREG2 is a FLEx cassette comprises a primer recognition sequence.

### FIELD OF THE INVENTION

The present invention relates to constructs, vectors and methods for carrying out parallel screening of gene regulatory elements in cells, tissues and in vivo. The invention also extends to novel pGREs identified using the constructs, vectors and methods.

### BACKGROUND TO THE INVENTION

Vectors for delivery of recombinant DNA, for example for gene therapy, take many forms, including viral and non-viral vectors. Viral vectors are gene delivery vehicles for delivering genetic material into a cell by using a viral genome and viral packaging (e.g. the viral capsid), while non-viral vectors are based on delivering genetic material into a cell by using inorganic particles, lipid-based vectors, polymer-based vectors, and peptide-based vectors. Furthermore, viral vectors are biologically based, while nonviral vectors are chemically based. Example non-viral vectors include lipid nano emulsions, polymethacrylate, dendrimers and the like. Example viral vectors include lentivirus, adenovirus, tobacco mosaic virus and adeno-associated virus.

Adeno-associated viruses (AAVs) have become the de facto viral vector for gene therapy applications. Their combination of excellent safety profile and relative ease of engineering has meant that they have found use in a wide range of disorders throughout tissue and cells types. AAVs contain two major components, a protein capsid and the AAV genome. Recombinant AAVs used for therapeutic and research purposes typically only retain one element of the genome that is native to wild type AAVs, the inverted terminal repeats (ITRs). Between the ITRs, the wild type genome is removed and replaced with non-native nucleotides which traditionally contain two further elements: the gene of interest and a regulatory sequence. The regulatory sequence contains promoter and/or enhancer sequences, which dictate the strength of gene expression and cell type in which the gene is expressed.

Traditionally, AAV gene therapy vectors have used ubiquitous regulatory elements that drive high levels of gene expression in all (or most) cell and tissue types. This approach has drawbacks where it is desirable to have gene expression targeted to a particular cell or tissue type. This is particularly important when gene expression in off-target cell types could have detrimental effects.

Numerous efforts have been made to identify regulatory elements that drive gene expression in a cell type-specific manner. However, AAV's relatively small packing capacity (4.9 Kb) compared to the size of regulatory elements within the genome, coupled with difficulties in identifying which regulatory elements drive cell specific expression, have meant there are only a limited number of these “short” regulatory elements suitable for use in AAV vectors.

Several attempts have been made to address this through the screening of regulatory elements in AAV vectors. Initially, potential regulatory sequences are identified through a combination of next generation sequencing, such as ATAC-seq or DNAse-seq, which selectively digest accessible chromatin (that is not protected by methylation) in the targeted cell type, followed by next generation sequencing to identify putative regulatory elements within that cell type. Bioinformatics analysis is then used to identify the most promising candidates which are then further analysed using biological screening. These in silico methods are often trained on data sets and can, consequently, exhibit bias.

Though advances in sequencing methods have led to an explosion in the number of identified sequences that could potentially drive cell type-specific gene expression in AAV vectors, the biological screening of these sequences is a significant bottleneck. In general, biological screening is carried out in one of two ways, serial or parallel:

Serial screening is performed by using individual sequences of regulatory elements upstream of a reporter construct, such as a fluorescent protein, followed by injection of the vectors into experimental animals and histological identification of cell types expressing the reporter. This method has been successfully used to identify highly specific regulatory elements, for example, using hecticetic promoters in the retina (Juttner et al., 2019), or enhancer sequences in primate neocortex (Mich et al., 2021). However, the identification of functional regulatory sequences requires that each is tested in an individual experimental animal. As 10 s or 100 s of sequences may need to be tested to find an appropriate regulatory sequence, this method is not scalable or practical.

Attempts have been made to parallelise the screening of regulatory element sequences in individual animals by placing the sequences upstream of a DNA barcode. This has the advantage that multiple sequences can be tested in a single experimental animal and has been successfully used to identify cell type-specific enhancer sequences for interneuron subtypes in the visual cortex (Hrvatin et al., 2019). However, limitations in sequencing technology and difficulties with mapping the DNA barcode to the correct regulatory element also limit this technique to <200 sequences being tested at one time. Additionally, use of experimental animals is complex and often undesirable.

Given the plethora of available sequencing data and potential useful regulatory elements a high throughput method for screening regulatory elements for their ability to drive cell type-specific gene expression is desirable for the development of cell or tissue targeted gene therapies. It is against this backdrop that the present invention has been devised. The invention describes a method for the high throughput screening of regulatory elements across multiple cell and tissue types. It can be adapted for use in cell culture systems and in animal models.

### SUMMARY OF THE INVENTION

According to a first aspect there is provided a nucleotide construct for use in a method of screening multiple putative gene regulatory elements (pGRE) in parallel, comprising nucleotide blocks, in sequence PRIMREG1-pGRE-PRIMREG2-GENE, or GENE-PRIMREG1-pGRE-PRIMREG2
wherein:
PRIMREG1 is either a primer recognition sequence 1, or a FLEx cassette comprising a primer recognition sequence 1.
pGRE is a putative gene regulatory element,
PRIMREG2 is either a primer recognition sequence 2, or a FLEx cassette comprising a primer recognition sequence 2,
GENE comprises nucleotide blocks REPSEQ, IRES and SSR, wherein
   REPSEQ is a reporter gene and is either present or absent,
   IRES is either an internal ribosome entry site or a 2A self-cleaving peptide, and
   SSR is a site specific recombinase gene;

wherein at least one of PRIMREG1 and PRIMREG2 is a FLEx cassette comprising a primer recognition sequence.

Advantageously, the nucleotide construct of the present invention allows for large pGRE libraries to be screened in parallel. By inserting a pGRE into a nucleotide construct that can generate a different PCR product dependent on primer design, active and inactive pGREs can be identified on a large scale.

In a second aspect there is provided a vector comprising a nucleotide sequence for use in a method of screening multiple putative gene regulatory element (pGRE) in parallel, comprising the nucleotide blocks, in sequence PRIMREG1-pGRE-PRIMREG2-REPSEQ-IRES-SSR, or GENE-PRIMREG1-pGRE-PRIMREG2
wherein:
PRIMREG1 is either a primer recognition sequence 1, or a FLEx cassette comprising a primer recognition sequence 1.
pGRE is a putative gene regulatory element,
PRIMREG2 is either a primer recognition sequence 2, or a FLEx cassette comprising a primer recognition sequence 2,
GENE comprises nucleotide blocks REPSEQ, IRES and SSR, wherein
   REPSEQ is a reporter gene and is either present or absent,
   IRES is either an internal ribosome entry site or a 2A self-cleaving peptide, and
   SSR is a site specific recombinase gene;
wherein at least one of PRIMREG1 and PRIMREG2 is a FLEx cassette comprising a primer recognition sequence.

The presently disclosed construct and method can be applied to any suitable DNA-based vector, allowing considerable flexibility.

In a third aspect there is provided a method of screening multiple putative gene regulatory elements (pGREs) in parallel comprising the steps:
a. generating or having generated a population of vectors comprising a nucleotide construct comprising nucleotide blocks, in sequence PRIMREG1-pGRE-PRIMREG2-GENE, or GENE-PRIMREG1-pGRE-PRIMREG2
wherein:
PRIMREG1 is either a primer recognition sequence 1, or a FLEx cassette comprising a primer recognition sequence 1.
pGRE is a putative gene regulatory element,
PRIMREG2 is either a primer recognition sequence 2, or a FLEx cassette comprising a primer recognition sequence 2,
GENE comprises nucleotide blocks REPSEQ IRES and SSR, wherein
   REPSEQ is a reporter gene,
   IRES is either an internal ribosome entry site or a 2A self-cleaving peptide, and
   SSR is a site specific recombinase gene;
wherein at least one of PRIMREG1 and PRIMREG2 is a FLEx cassette comprising a primer recognition sequence;
b. contacting target cells with the population of AAV vectors under conditions suitable for transduction of the target cells;
c. isolating DNA from the transduced target cells;
d. selectively amplifying active and/or inactive pGREs using PCR; and
e. determining the sequence of active and/or inactive pGREs.

### BRIEF DESCRIPTION OF THE DRAWINGS

For a better understanding of the invention and to show how the same may be carried into effect, there will now be described by way of example only, specific embodiments, methods and processes according to the present invention with reference to the accompanying drawings in which:

FIG. 1 shows an example nucleotide construct suitable for use in an AAV vector. The GENE block is shown downstream (3′) of the pGRE and is indicated in the order REPSEQ-IRES-SSR.

FIG. 2 shows an inactive pGRE using the construct of FIG. 1. The PCR product is generated using specific primers to the non-inverted primer recognition sequence.

FIG. 3 shows an active pGRE using the construct of FIG. 1. The PCR product is generated using specific primers to the inverted primer recognition sequence.

FIG. 4 shows a schematic of the method in which 1. Indicates pGRE library generation, showing multiple different pGREs in individual nucleotide constructs. 2. Indicated the constructs from 1 being packaged into a vector, such as AAV. 3. Indicates transduction of cells/tissues of interest by the vectors. 4. Indicates harvesting of DNA from the transduced cells/tissue. 5. Indicates PCR using forward and reverse primers (selected according to whether active or inactive pGREs are being screened for). 6. Indicates sequencing of PCR product.

FIG. 5A shows an example method using AAV and screening in neuro2A cells. FIG. 5B shows the output from the method of FIG. 5A.

### DETAILED DESCRIPTION

Nucleotide construct as employed herein refers to a synthetic or recombinant double stranded DNA construct comprising the nucleotide blocks defined below. Typically, nucleotide constructs are described in sequence, that is, in the 5′ to 340 direction. However, it will be appreciated that block order is not always strictly necessary. For example, 3′ pGREs can be screened for by changing the order of the blocks so that the genes (SSR and REPSEQ) are positioned upstream of the pGRE and FLEx cassette.

Method of screening multiple putative gene regulatory elements (pGREs) in parallel as employed herein refers to the scalability of the described method. The method advantageously employs libraries of pGREs which can then be used to generate many different nucleotide constructs. The library of constructs is then used to generate a vector library. The vectors are then introduced to a population of cells, or a tissue sample or even an animal, where they transduce cells and can, subsequently, be identified as either active or inactive in that cell-type.

Nucleotide blocks as employed herein refers to a defined segment of the nucleotide construct that performs a particular function within the construct. Nucleotide blocks may be contiguous or may be distally spaced, for example, by nucleotides which may perform a function or may be linker nucleotides.

In sequence as employed herein means that the nucleotide sequence is read in the 5′ to 3′ direction by convention.

ITR1 as employed herein refers to the 5′ inverted terminal repeat of the nucleotide construct.

Inverted terminal repeats are inverted repeats that occur at the termini of the nucleotide construct. The inverted repeat is a sequence of nucleotides followed, downstream, i.e. at the 3′ end, by its reverse complement.

PRIMREG1 as employed herein refers to the 5′ primer recognition sequence block. 5′ in this context means 5′ (upstream) relative to the pGRE. 5′ also refers to the primer recognition sequence block closest to the 5′ end of the nucleotide construct. The block comprises a nucleotide sequence against which PCR primers can be designed to enable amplification of active and/or inactive pGREs. The primer recognition sequence of the PRIMREG1 block may be or comprise a FLEx cassette.

pGRE as employed herein means putative gene regulatory element. The term is intended to encompass promoters, enhancers and silencers of gene expression.

PRIMREG2 as employed herein refers to the 3′ primer recognition sequence block. 3′ in this context means 3′ (downstream) relative to the pGRE. 3′ also refers to the primer recognition sequence block closest to the 3′ end of the nucleotide construct. The block comprises a nucleotide sequence against which PCR primers can be designed to enable amplification of active and/or inactive pGREs. The primer recognition sequence of the PRIMREG2 block may be or comprise a FLEx cassette.

At least one of PRIMREG1 and PRIMREG2 is a FLEx cassette. In general, it is only necessary for one for PRIMREG1 and PRIMREG2 to be a FLEx cassette.

GENE as employed herein refers to the block comprising REPSEQ, IRES and SSR blocks as described herein.

REPSEQ as employed herein refers to a reporter gene. Suitable reporter genes will be known to the skilled person but include, without limitation, green fluorescent protein (GFP), blue fluorescent protein (BFP), mCherry, mScarlet or bioluminescent reporter genes such as recombinant firefly luciferase or NanoLuc Luciferase. Other genes that can be tested for may act as reporters even where they are not fluorescent/luminescent. Yet other nucleotide sequences that could be detected, for example, by PCR, may be used as reporter genes. It will be appreciated that, although useful experimentally, the reporter gene is not needed in all circumstances. Thus, the REPSEQ block may be present or absent.

IRES as employed herein refers to an internal ribosome entry site. The site may be present or absent. Alternatively, the IRES block may be a 2A self-cleaving peptide and may be present or absent. Typically, either an IRES or a 2A self-cleaving peptide (or a suitable equivalent) will be present.

2A self-cleaving peptide as employed herein is a class of 18-22 aa-long peptides, which can induce ribosomal skipping during translation of a protein in a cell. These peptides share a core sequence motif of DxExNPGP, and are found in a wide range of viral families.

SSR as employed herein means site specific recombinase. Site specific recombinases are enzymes that perform rearrangements of DNA segments by recognising and binding to short, specific DNA sequences (sites) at which they cleave the DNA backbone, exchange the two DNA strands involved and rejoin them. Suitable SSRs include, but are not limited to cre recombinase (cre), Flp/FLP (flippase), Dre (D6 recombinase). SSRs bind DNA at target sites to induce site specific recombination events, for example, Cre recombinase binds loxP sites, while FLP binds FRT sites and Dre binds Rox sites.

ITR2 as employed herein refers to the 3′ inverted terminal repeat of the nucleotide construct.

FLEx cassette as employed herein refers to flip-excision switches with a primer recognition sequence flanked by SSR recognition sites. FLEx switches were designed as a genetic tool for researchers to conditionally manipulate gene expression in vivo using site-specific recombination. The FLEx switch takes advantage of the orientation specificity of SSRs such as Cre and FLP. SSRs bind DNA at target sites to induce site specific recombination events. When a DNA sequence is flanked by target sites (floxed) in opposing orientations, a SSR will invert the DNA sequence between the sites. In this case, the primer recognition sequence.

By way of example, a FLEx switch that turns BFP expression off, while turning on mCherry expression could be achieved by creating a cassette with BFP coding sequence in the sense orientation and mCherry coding sequence in the antisense orientation. The DNA would be flanked (floxed) by two pairs of target sites for example, one wild-type pair (e.g. loxP) and one mutated pair (e.g. lox511). It is necessary to use two different pairs of target sites for this strategy to work effectively. Both loxP and lox511 are recognised by Cre but lox511 sites can only recombine with other lox511 sites, not with loxP sites. Alternatively, you could use a pair of loxP sites and a pair of FRT sites and include both Cre and FLP recombinases if construct size is not of concern. The selection of target sites is not restricted, provided that two different pairs are provided.

Once the SSR of choice is introduced or activated by an active pGRE in the present invention, recombination can proceed either by first utilising the loxP sites or the lox551 sites. Regardless, the first recombination step will invert the intervening DNA fragment using either loxP or lox511 sites, leaving two identical sites on one end of the DNA fragment (either 3′ or 5′ of the BFP/mCherry DNA). A second recombination event then excises the DNA between the identical loxP or lox511 sites, leaving only one loxP and lox511 site on either side of the DNA fragment. Any additional recombination events are impossible at this stage, even in the presence of Cre recombinase. This plasmid would now specifically drive expression of mCherry instead of BFP.

In the present method, the reporter sequence REPSEQ (e.g. BFP) has been moved outside of the FLEx cassette such that it is expressed in the presence of an active pGRE and not expressed in the presence of an inactive pGRE. Similarly, expression of the SSR is driven by an active pGRE and not expressed where the pGRE is inactive. In contrast to the example above, the FLEx cassette comprises of a primer recognition sequence floxed by a pair of SSR recognition sites. When the SSR is expressed, i.e. the pGRE is active, the primer recognition sequence is inverted. PCR primers can then be designed to amplify active/flipped DNA or inactive/unflipped DNA.

Primer recognition sequence as employed herein means a short sequence (typically approximately 18 to 24 bases) which is unique to the nucleotide construct and to which a PCR primer sequence, complementary to it, can specifically bind. Primer design will be known to the skilled person, therefore suitable primer recognition sequences will follow equivalent parameters.

Vector as employed herein may be a viral or a non-viral vector.

Suitable non-viral vectors include, but are not limited to, using inorganic particles, lipid-based vectors, polymer-based vectors, and peptide-based vectors.

Suitable viral vectors include any suitable DNA-based virus including, but not limited to, AAV, lentivirus, adenovirus, and tobacco mosaic virus.

AAV vector as employed herein are adeno-associated virus viral particles comprising a nucleotide construct packaged within an AAV viral capsid that is capable of tranducing a cell. The AAV capsid may be capable of specifically infecting some cell types in preference to other cell types.

AAVs are small viruses belonging to the genus dependoparvovirus containing a single strand of DNA, up to ~4.9 Kb. The AAV genome contains three capsids proteins VP1, VP2 and VP3, all of which are translated from one mRNA via alternate splicing. In the wild, multiple serotypes of AAV have been identified each with unique sequences of capsid gene, and hence distinct tropisms, although wild serotypes tend to be able to infect multiple tissue and cell types. These serotypes are denoted by numbers: AAV1, AAV2, etc. It has been shown that modification of capsid sequences via DNA recombination methods can generate non-native sequences with tailored properties and tropism directed towards (or against) particular cells or tissues, and that evade the immune system (Vandenberghe et al., 2009).

Generating or having generated a population of vectors, such as AAV vectors, as employed herein means either obtaining a pre-prepared population of vectors from a suitable source or generating them at the time of performing the method disclosed herein. Typically, generating the population of vectors comprises the steps: generating a library of pGREs using any suitable methodology, using molecular biology techniques to generate nucleotide constructs according to the present invention wherein the pGRE blocks are those from the pGRE library, cloned into an appropriate vector genome (such as an AAV genome flanked by ITRs), and packaging the vector genome into a suitable vector, such as an AAV capsid, to generate the population of AAV vectors.

pGRE libraries may be generated by any suitable method, including, but not limited to, oligo printing of rationally/bioinformatically designed constructs, DNA pulled from ATAC-seq or DNAse-seq protocols which selectively digest accessible chromatin in the targeted cell type, random mutagenesis or DNA shuffling of previously published pGREs or previous pGREs libraries.

Where ATAC-seq libraries are employed, the requirement to generate candidates in silico is replaced by the biological method disclosed herein. Advantageously, this means that any potential bias introduced by the in silico training data is removed and the potential to identify novel pGREs is increased.

Contacting target cells as employed herein means that the vector population is introduced to the target cells using a method suitable for the vector and the cell type to be transduced. This will vary depending on whether the target cells are cells in culture, tissues or organoids, or model animals.

Conditions suitable for transduction of the target cells as employed herein refers to the experimental conditions and will vary depending on the target cells. For example, the conditions in cell culture may involve controlling the temperature, pH and nutrients available. In contrast, in an animal model, the conditions may be met simply by injection of the vectors into the host animal.

It is important that only one vector transduces each cell because, for example, if two vectors (one active and one inactive) transduced the same cell, the SSR would act on both constructs (the active AND the inactive one) leading to a false identification of an inactive pGRE as active.

Where the vector is a non-viral vector, conditions suitable for transfection of the target cells will vary depending on the target cells and transfection protocol. For example, the conditions in cell culture for electroporation transfection may involve applying an electric field to form pores in the cell membrane and allow the molecule to pass. In contrast, for a chemical carrier transfection protocol, the conditions may require inorganic nanoparticles, such as gold or supermagnetic iron oxide coated to facilitate DNA binding.

Isolating DNA from the transduced cells as employed herein refers to the process of extracting the DNA from the transduced cells. In general, the three basic steps involved will be lysis of the cells, precipitation of the DNA, and purification of the precipitate. These techniques are well known in the field of molecular biology but not intended to be limiting on the scope of the methodology since all suitable methods would be applicable.

Selectively amplifying as employed herein refers to the process by which a nucleic acid molecule is copied to generate multiple copies of the same sequence. The most widely used method for amplifying nucleic acid sequences is PCR (polymerase chain reaction). The method described herein employs PCR techniques to amplify active and/or inactive pGREs but it will be appreciated that other methods, including not amplifying the pGREs at all, could be employed without deviating from the spirit of the invention. For example, the entire nucleotide construct, or a part thereof, could be sequenced without amplification.

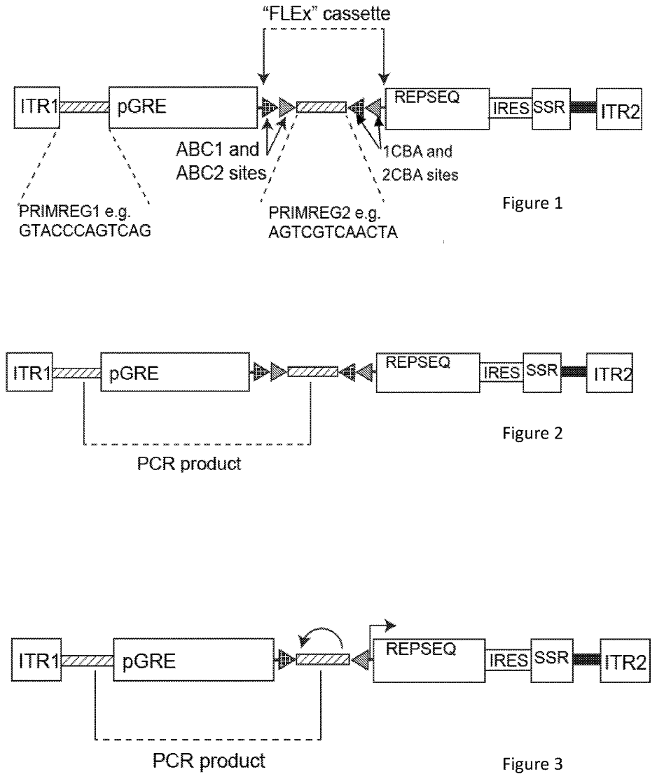

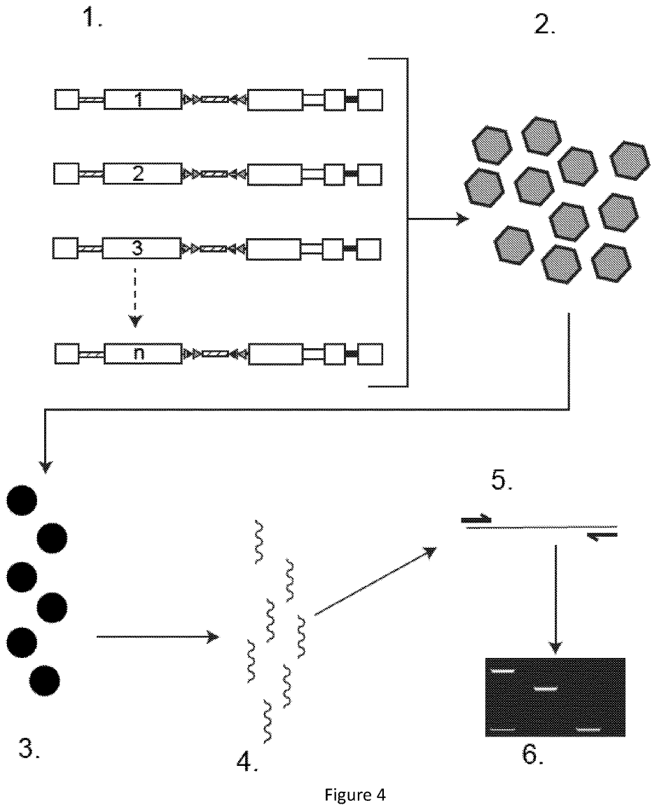

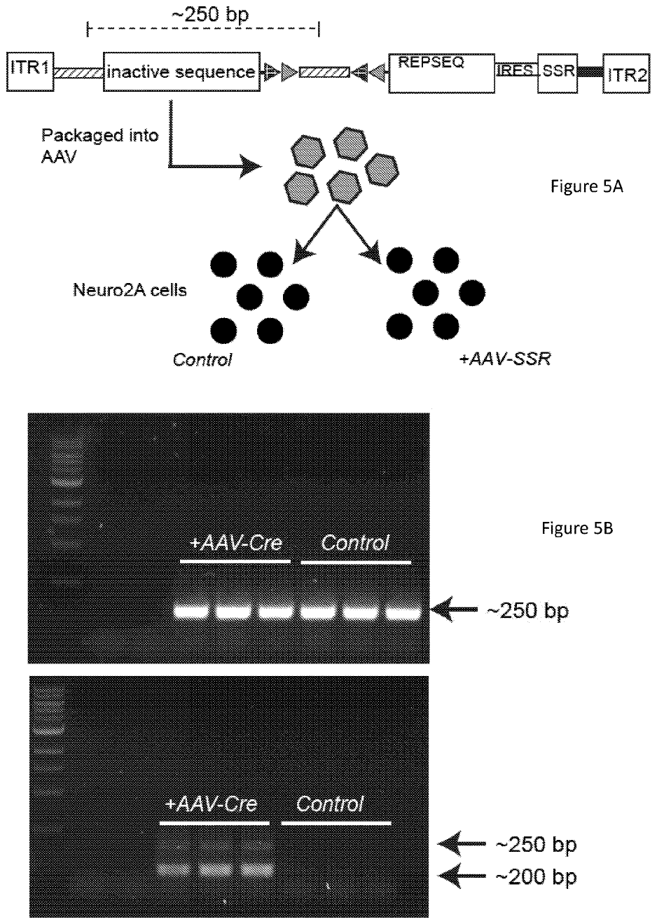

Active pGREs as employed herein refers to pGREs that switch on expression of the genes of the nucleotide construct, such as the reporter gene and/or the SSR gene (encoded by the REPSEQ and SSR blocks respectively. Active pGREs can be identified by the fact that the primer recognition sequence of the FLEx cassette has been inverted. Where PCR methods are employed, the amplified nucleotide sequence that is produced is dependent on the selection of primers. If primers to both inactive and active pGREs are used, the active pGREs produce an amplified nucleotide sequence that will be shorter than that produced when the primer recognition sequence is not inverted (i.e. when the pGRE is inactive). This is because one of the 5′ SSR recognition sites is excised by the site specific recombinase, e.g. ABC1 or ABC2 (See FIG. 3). Furthermore, the primer choice enables either active or inactive (or both) pGREs to be selectively amplified. Example primers to the example primer recognition sequences shown in FIG. 1 would be:

Forward primer: GTACCCAGTCAG (binds to the 5′ primer recognition sequence)

Reverse primer 1: TAGTTGACGACT (binds to the 3′ primer recognition sequence when pGRE is inactive)

Reverse primer 2: AGTCGTCAACTA (binds to the 3′ primer recognition sequence when pGRE is active and the flex cassette is inverted).

It will be appreciated that the primer sequences are not limited by the foregoing example.

Inactive pGREs as employed herein refers to pGREs that do not switch on expression of the genes of the nucleotide construct, such as the reporter gene and/or the SSR gene (encoded by the REPSEQ and SSR blocks respectively. Inactive pGREs can be identified by the fact that the primer recognition sequence of the FLEx cassette has not been inverted. Where PCR methods are employed, the amplified nucleotide sequence is that is produced is dependent on the selection of primers. If primers to both inactive and active pGREs are used, the amplified nucleotide sequence that is produced will be longer than that produced when the primer recognition sequence is inverted (i.e. when the pGRE is active). This is because none of the 5′ SSR recognition sites are excised by the site specific recombinase, e.g. both ABC1 and ABC2 are still present (See FIG. 2). Furthermore, the primer choice enables either active or inactive (or both) pGREs to be selectively amplified.

Determining the sequence as employed herein refers to any suitable method of sequencing the amplified nucleotide sequences. Once the sequence of active pGREs is known, they can be further investigated. Similarly, pattern in inactive pGREs could be identified utilising bioinformatics techniques.

ABC1 as employed herein refers to the first SSR recognition site. This is a site recognised by the SSR encoded by the SSR block. SSR cleaves the DNA at the ABC1 site and reattaches it to the 1CBA site during the method. The ABC1 block will be different to the ABC2 block.

ABC2 as employed herein refers to the second SSR recognition site. This is a site recognised by the SSR encoded by the SSR block. SSR cleaves the DNA at the ABC2 site and reattaches it to the 2CBA site during the method. Although the method employs the same SSR for both inversions, different SSRs could be employed.

1CBA is an inverted copy of ABC1, located downstream (3′) of the primer recognition sequence.

2CBA is an inverted copy of ABC2, located downstream (3′) of the primer recognition sequence.

SSR inversion is a two-step process. One inversion at the ABC1 and 1CBA sites, and another inversion at the ABC2 and 2CBA sites. The order in which ABC1-1BCA and ABC2-2CBA inversions occur is not important. The first inversion will invert the primer recognition sequence, leaving one of the SSR recognition sites on either the 5′ or 3′ side of the primer recognition sequence, and 3 SSR recognition sites on the other side. The second inversion will excise two of the SSR recognition sites, leaving one of the ABC1-1CBA pair and one of the ABC2-2CBA pair behind. Because of this, further inversions by SSR cannot occur.

PRSEQ is the primer recognition sequence within the FLEx cassette.

Lox1 as employed herein is a Lox sequence, which comprises a site recognised by cre SSR. 1xoL is the inverted copy of Lox1, located downstream of PRSEQ.

Lox2 as employed herein is a different Lox sequence with a different site recognised by cre SSR. 1xoL is the inverted copy of Lox2, located downstream of PRSEQ.

FRT1 as employed herein is a FRT sequence, which comprises a site recognised by FLP SSR. 1TRF is the inverted copy of FRT1, located downstream of PRSEQ.

FRT2 as employed herein is a different FRT sequence with a different site recognised by FLP SSR. 2TRF is the inverted copy of FRT2, located downstream of PRSEQ.

Rox1 as employed herein is a Rox sequence, which comprises a site recognised by Dre SSR. 1xoR is the inverted copy of Rox1, located downstream of PRSEQ.

Rox2 as employed herein is a Rox sequence, which comprises a site recognised by Dre SSR. 2xoR is the inverted copy of Rox2, located downstream of PRSEQ.

### Methodology

First, a population of nucleotide constructs are created containing a library of putative gene regulatory elements (pGREs), such as promoters and enhancers. Each individual nucleotide construct contains one pGRE. Multiple constructs are combined and packaged into recombinant AAV virions to create a population of AAV vectors containing a pGRE library. Each virion contains one construct. This library of pGREs can contain sequences generated through rational design or by direct amplification from a mammalian genome such as via the tagmentation with Tn5 transposase as is used in methods such as ATAC-Seq.

In the nucleotide construct, the pGRE sequence is flanked by a region of DNA that binds a PCR primer (primer recognition sequence). A primer recognition sequence is found both 5′ and 3′ to the pGRE. One (or more) of the primer recognition sequences are flanked by two sets of heterotypic antiparallel recombinase recognition sites, such as LoxP sites, in a combination sometimes known as a flip-excision (FLEx) switch. When a site specific recombinase, such as cre, acts on this region the DNA between the recognition sites flips orientation, then excises the now parallel LoxP sites, preventing any further flipping of the orientation. (see FIG. 3).

The pGRE region is upstream from the SSR to act on the recognition sites, such as cre and LoxP. In the case of cre-LoxP if the pGRE region is sufficient to drive expression of the cre gene, cre protein is produced causing recombination of the LoxP sites and inversion of the primer recognition sequence.

Pairs of PCR primers can be designed such that they recognise the primer recognition sequences in either the unrecombined (inactive) or recombined (active) case, thus allowing the selective PCR of active and inactive pGREs in separate PCR reactions.

Transduction of a rAAV pGRE library into cells in culture, or whole tissues in vivo, allows the screening of a large number of pGREs in parallel. In this case, harvesting of the population of cells followed by PCR will generate a PCR product which can be subjected to next generation or Sanger sequencing to ultimately ascertain the sequence of active (and inactive) GREs across cell and tissue types.

In an extension of this method multiple (both) primer recognition sequences can be flanked (floxed) by recombinase recognition sites. For example, the 5′ primer recognition sequence could be flanked by FRT sites (recognised by FLP recombinase), while the 3′ primer recognition sequence is flanked by LoxP site (recognised by cre); or visa versa. Other recombinases and recognition sites such as Dre-Rox could also be used. In this case, a certain combination of PCR primers could be used to detect pGREs where both cre and flp recombinases are present.

One example use for this would be for the assay of pGREs in recombinase-expressing animals such as cre transgenic mice. Here the nucleotide construct would encode for FLP-recombinase required for the inversion of one priming region and the target cell type would provide the cre for the inversion of the other priming region. For example, injection of this vector into ChAT-Cre experimental mice, which express cre in cholinergic cells such as motor neurons, would allow the selective harvesting and identification by PCR of pGREs which are active in motor neurons.

pGREs which give positive results from these types of screen can be subjected to further analysis such as via individual screening or via DNA-barcoding methods.

Approximately as employed herein means ±10%.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

### REFERENCES

Juttner et al., 2019 Targeting neuronal and glial cell types with synthetic promoter AAVs in mice, non-human primates and humans. Nature Neuroscience volume 22, pages 1345-1356 (2019)
Mich et al., 2021 Functional enhancer elements drive subclass-selective expression from mouse to primate neocortex. Cell Reports 34, 108754
Hrvatin et al., 2019 A scalable platform for the development of cell-type-specific viral drivers. eLife 8: e48089

1. A nucleotide construct for use in a method of screening multiple putative gene regulatory elements (pGRE) in parallel, comprising nucleotide blocks, in sequence PRIMREG1-pGRE-PRIMREG2-GENE, or GENE-PRIMREG1-pGRE-PRIMREG2 wherein:
PRIMREG1 is either a primer recognition sequence 1, or a FLEx cassette comprising a primer recognition sequence 1.
pGRE is a putative gene regulatory element,
PRIMREG2 is either a primer recognition sequence 2, or a FLEx cassette comprising a primer recognition sequence 2,
GENE comprises nucleotide blocks REPSEQ, IRES and SSR, wherein
REPSEQ is a reporter gene and is either present or absent,
IRES is either an internal ribosome entry site or a 2A self-cleaving peptide, and
SSR is a site specific recombinase gene;
wherein at least one of PRIMREG1 and PRIMREG2 is a FLEx cassette comprising a primer recognition sequence.

2. A vector comprising a nucleotide sequence for use in a method of screening multiple putative gene regulatory element (pGRE) in parallel, comprising the nucleotide blocks, in sequence PRIMREG1-pGRE-PRIMREG2-REPSEQ-IRES-SSR, or GENE-PRIMREG1-pGRE-PRIMREG2
wherein:
PRIMREG1 is either a primer recognition sequence 1, or a FLEx cassette comprising a primer recognition sequence 1.
pGRE is a putative gene regulatory element,
PRIMREG2 is either a primer recognition sequence 2, or a FLEx cassette comprising a primer recognition sequence 2,
GENE comprises nucleotide blocks REPSEQ, IRES and SSR, wherein
REPSEQ is a reporter gene and is either present or absent,
IRES is either an internal ribosome entry site or a 2A self-cleaving peptide, and
SSR is a site specific recombinase gene;
wherein at least one of PRIMREG1 and PRIMREG2 is a FLEx cassette comprising a primer recognition sequence.

3. A method of screening multiple putative gene regulatory elements (pGREs) in parallel comprising the steps:
a. generating or having generated a population of vectors comprising a nucleotide construct comprising nucleotide blocks, in sequence PRIMREG1-pGRE-PRIMREG2-GENE, or GENE-PRIMREG1-pGRE-PRIMREG2
wherein:
PRIMREG1 is either a primer recognition sequence 1, or a FLEx cassette comprising a primer recognition sequence 1.
pGRE is a putative gene regulatory element,
PRIMREG2 is either a primer recognition sequence 2, or a FLEx cassette comprising a primer recognition sequence 2,
GENE comprises nucleotide blocks REPSEQ IRES and SSR, wherein
REPSEQ is a reporter gene,
IRES is either an internal ribosome entry site or a 2A self-cleaving peptide, and
SSR is a site specific recombinase gene;
wherein at least one of PRIMREG1 and PRIMREG2 is a FLEx cassette comprising a primer recognition sequence;
b. contacting target cells with the population of AAV vectors under conditions suitable for transduction of the target cells;
c. isolating DNA from the transduced target cells;
d. selectively amplifying active and/or inactive pGREs using PCR, and
e. determining the sequence of active and/or inactive pGREs.

4. The vector according to claim 2 wherein the vector is a viral vector.

5. The vector or method according to claim 4, wherein the viral vector is an AAV vector.

6. The construct, vector or method according to claim 1 wherein the FLEx cassette comprises, in sequence, ABC1-ABC2-PRSEQ-1CBA-2CBA wherein:
ABC1 is a recognition site for the recombinase protein encoded by the SSR block,
ABC2 is a different recognition site for the recombinase protein encoded by the SSR block,
PRSEQ is either primer recognition sequence 1 or primer recognition sequence 2,
1CBA is inverted ABC1, and
2CBA is inverted ABC2.

7. The construct, vector or method according to claim 6 wherein:
ABC1 is Lox1,
ABC2 is Lox2,
SSR is cre,
1CBA is 1xoL, and
1CBA is 2xoL.

8. The construct, vector or method according to claim 6 wherein:
ABC1 is FRT1,
ABC2 is FRT2,
SSR is Flp,
1CBA is 1TRF, and
1CBA is 2TRF.

9. The construct, vector or method according to claim 6 wherein:
ABC1 is Rox1,
ABC2 is Rox2,
SSR is dre,
1CBA is 1xoR, and
1CBA is 2xoR.

10. The construct, vector or method according to claim 1 wherein the nucleotide construct has a 5′ ITR1 block and a 3′ ITR2 block.

11. The method according to claim 3 wherein both PRIMREG1 and PRIMREG2 are FLEx cassettes, and the target cells are capable of expressing a site specific recombinase which is different to the one encoded by the SSR block of the nucleotide construct.

12. A novel pGRE identified using the method of claim 3.

13. The method according to claim 3 wherein the vector is a viral vector.
